(19) United States
(12) Patent Application Publication
(10) Pub. No.: US 2009/0054329 A1
(43) Pub. Date: Feb. 26, 2009

(54) COMPOSITION WITH DOCOSAPENTAENOIC ACID

(75) Inventors: Linette Eustachia Maria Willemsen, Utrecht (NL); Eric Alexander Franciscus Van Tol, Arnhem (NL); Christopher Beermann, Neu-Anspach (DE)

Correspondence Address: FOLEY AND LARDNER LLP;SUITE 500, 3000 K STREET NW, WASHINGTON, DC, 20007, US

(73) Assignee: N.V. Nutricia

(21) Appl. No.: 12/094,130

(22) PCT Filed: Nov. 16, 2006
(86) PCT No.: PCT/NL2006/050291

§ 371 Date: Sep. 8, 2008

(30) Foreign Application Priority Data
Nov. 17, 2005 (NL) PCTNL2005050044

(51) Int. Cl. A61K 38/16; A61K 31/202; A61K 31/715; A61P 1/14

(52) U.S. Cl. 514/12; 514/560; 514/53

(57) ABSTRACT

This invention concerns a method for stimulating barrier integrity in a subject by administering to a subject a composition comprising docosapentaenoic acid (22:5 n3; DPA). Also the invention concerns a composition comprising DPA and eicosapentaenoic acid (EPA).

### FIELD OF THE INVENTION

The present invention relates to nutritional and pharmaceutical compositions with long chain polyunsaturated fatty acids.

### BACKGROUND OF THE INVENTION

Long chain polyunsaturated fatty acids (LCPUFA) are often included in nutritional composition for their beneficial effects. Particularly in infant formulas, LCPUFA such as arachidonic acid and docosahexaenoic acid are included in infant formulas which aim to improve cognitive functions and vision.

Eicosapentaenoic acid (EPA, 20:5 n3) is another important LCPUFA. Fish oil contains relatively high quantities of EPA compared to for example vegetable oils. However, fish oil has the disadvantage that often contributes to an off-smell of the nutritional product. Complex technological measures are required to resolve this problem.

### SUMMARY OF THE INVENTION

The present inventors have found that LCPUFA can stimulate barrier integrity. In applicants co-pending European patent application (appl. number 04748674.1), it was described that a combination of indigestible oligosaccharide and LCPUFA (arachidonic acid (20:4 n6; AA), docosahexaenoic acid (22:6 n3; DHA) and eicosapentaenoic acid (20:5 n3; EPA)) improve the barrier integrity.

The present inventions have now found that besides AA, DHA and EPA, also docosapentaenoic acid (22:5 n3; DPA) strongly reduces barrier permeability and increases barrier integrity of human intestinal epithelial cells. These effects are particularly important in those situations where the intestinal barrier function is impaired or where maturation of the intestinal tract is important. Hence, in one aspect the present invention provides the use of DPA for stimulating the maturation of the intestine, reducing permeability of the intestinal tract and increasing intestinal barrier resistance.

Similar to the invention described in applicants' co-pending application (appl. number 04748674.1), the present invention also provides the combination of indigestible oligosaccharides and DPA, which synergistically strengthens the barrier function. It was surprisingly found that DPA effectively improves barrier integrity and reduces epithelial paracellular permeability, while the PUFAs linoleic and linolenic acid were found to be ineffective. The oligosaccharides further improve the barrier integrity by stimulating the production of the mucus, which results in an increased mucus layer thickness. It is believed this effect is caused by stimulated short chain fatty acid (SCFA) production. Hence, when enterally administered to a mammal, the present combination of DPA and indigestible oligosaccharides synergistically improves barrier integrity and/or synergistically reduces intestinal permeability by simultaneous reduction of tight junction permeability and stimulation of mucus production.

Furthermore, the present inventors have now also found that after incubation of human intestinal epithelial cells with DPA, part of the DPA was incorporated as EPA in the membrane phospholipids of the cells, indicating that DPA is partially converted to EPA in the epithelial cells. This observation enables the formulation of nutritional compositions in which at least part of the EPA is replaced by DPA. This provides the opportunity the manufacture nutritional compositions with reduced fish oil inclusion and thus with reduced off-flavor and smell.

The combined observations that DPA is highly effective in reducing barrier permeability and that part of the DPA is converted to EPA in epithelial cells now enables the formulation of nutritional compositions, particularly infant formula, with a reduced EPA content and an increased DPA content, i.e. a DPA:EPA ratio of about 0.33 or more. The present composition has an improved or at least equal functionality compared to high EPA compositions and a potentially reduced bad flavor because the fish oil content is reduced.

### DETAILED DESCRIPTION

The present invention provides the use of n-3 docosapentaenoic acid (DPA) for the manufacture of a nutritional or pharmaceutical composition for improving intestinal barrier integrity, improving barrier function, stimulating gut maturation and/or reducing intestinal barrier permeability. The invention also provides a method for improving intestinal barrier integrity, improving barrier function, stimulating gut maturation and/or reducing intestinal barrier permeability in a subject, said method comprising administering n-3 docosapentaenoic acid (DPA) to said subject.

In a further aspect the present invention provides the use of n-3 docosapentaenoic acid, for the manufacture of a nutritional or pharmaceutical composition for the treatment and/or prevention of diseases wherein barrier integrity is impaired. The invention also provides a method for the treatment and/or prevention of diseases wherein barrier integrity is impaired in a subject, said method comprising administering n-3 docosapentaenoic acid (DPA) to said subject.

In yet a further aspect the present invention provides a nutritional or pharmaceutical composition comprising n-3 docosapentaenoic acid (DPA) and eicosapentaenoic acid (EPA) wherein the ratio DPA:(EPA) is equal to or higher than 0.33.

### Polyunsaturated Fatty Acids

The present inventors surprisingly found that n-3 docosapentaenoic acid (DPA) effectively reduces intestinal tight junction permeability. The present inventors found that already low concentrations of DPA were effective. It is noted that DPA can relate to n3 and n6 family of PUFA. The present invention relates to the use of DPA n3, and whenever the abbreviation DPA is used, this refers to docosapentaenoic acid, 22:5 n3.

The composition used in the present method (hereinafter also referred to as present composition) preferably has a weight ratio DPA:EPA above about 1:3 (i.e. above about 0.33), more preferably above 1:2, more preferably above 1:1, more preferably above 1.2:1, even more preferably above 1.5:1 most preferably above 2:1. Most preferably, the weight ratio is between 1:1 and 10:1, more preferably between 2:1 and 5:1.

Preferably the present composition contains at least 10 mg DPA, preferably at least 50 mg, more preferably at least 100 mg, most preferably at least 250 mg. The present composition preferably does not contain more than 25 gram DPA, preferably not more than 1 gram DPA. These amounts are preferably administered in a single serving.

The present composition is preferably a nutritional composition, containing fat, lipids and carbohydrate. The DPA content of the present composition preferably does not exceed 5 wt. % of the total fat, more preferably does not exceed 1 wt. %, but is preferably at least 0.05 wt %, more preferably at least 0.1 wt. %, most preferably at least 0.15 wt. % of the total fat. Because the DPA is partially converted to EPA, the amount of EPA can be relatively low in the present composition. The EPA content preferably does not exceed 5 wt. % of the total fat, more preferably does not exceed 1 wt. %, most preferably does not exceed 0.5 wt. %. EPA is however advantageously present to improve barrier functionality. Hence, the present composition preferably comprises at least 0.02 wt. % EPA based on total fat, more preferably at least 0.05 wt. %, more preferably at least 0.1 wt. %.

For further improvement of the barrier integrity improving effects of the present composition, the present composition preferably also contains AA and/or DHA. Also these LC-PUFA improve barrier integrity. The DHA content preferably does not exceed 5 wt. %, more preferably does not exceed 1 wt. %, but is at least 0.1 wt % of the total fat. As AA was found to be particularly effective in reducing tight junction permeability, the present composition preferably comprises relatively high amounts of AA, preferably at least 0.1 wt. %, even more preferably at least 0.25 wt. %, most preferably at least 0.40 wt. % of the total fat. The AA content preferably does not exceed 5 wt. %, more preferably does not exceed 1 wt. % of the total fat. In the present AA containing enteral composition, EPA and DHA are advantageously added to balance the action of AA, e.g. reduce the potential pro-inflammatory action of AA metabolites. Excess metabolites from AA may cause inflammation. Hence, the present composition preferably comprises DPA, AA, EPA and DHA, wherein the weight ratio AA/DHA preferably is above 0.25, preferably above 0.5, even more preferably above 1. The ratio is preferably below 25. The weight ratio AA/EPA is preferably between 1 and 100, more preferably between 5 and 20.

To make the composition suitable for feeding to humans, preferably to infants, the content of LCPUFA with 20 and 22 carbon atoms in the present composition, preferably does not exceed 15 wt. % of the total fat content, preferably does not exceed 10 wt. %, even more preferably does not exceed 5 wt. % of the total fat content. Preferably the present composition comprises at least 0.1 wt. %, preferably at least 0.25 wt %, more preferably at least 0.5 wt. %, even more preferably at least 0.75 wt. % LC-PUFA with 20 and 22 carbon atoms of the total fat content.

The present composition preferably comprises between 5 and 75 wt. % polyunsaturated fatty acids based on total fat, preferably between 10 and 50 wt. %.

If the present composition is used as an infant formula (e.g. in a method for feeding an infant, said method comprising administering the present composition to an infant), the content of LC-PUFA, particularly the LC-PUFA with 20 and 22 carbon atoms, preferably does not exceed 3 wt. % of the total fat content as it is desirable to mimic human milk as closely as possible. For the same reason, the omega-3 LC-PUFA content preferably does not exceed 1 wt. % of the total fat content; the omega-6 LC-PUFA content preferably does not exceed 2 wt. % of the total fat content; the AA (omega-6) content is preferably below 1 wt. % of the total fat content; and/or the weight ratio EPA/DHA is preferably 1 or lower, more preferably below 0.5.

The LCPUFA with 20 and 22 carbon atoms are preferably provided as free fatty acids, in monoglyceride, diglyceride and/or triglyceride form, in phospholipid form, or as a mixture of one of more of the above. These sources are preferably interesterified to improve stability. The present composition preferably comprises at least one of AA and DHA in phospholipid and/or triglyceride form.

Preferably, the present composition comprises a fat source selected from the group consisting of plant fat, fungal fat or animal (excluding human) fat.

It was also found by the present inventors that DPA is capable of reducing the effects of IL-4 on intestinal permeability. Hence, in one aspect of the present invention provides a method for the treatment and/or prevention of diseases wherein intestinal IL-4 concentration is increased (e.g. allergic diseases), said method comprising administering the present DPA containing composition, preferably combined with the present indigestible oligosaccharides. Hence, the present composition can also be advantageously used in a method for the treatment and/or prevention of atopic dermatitis.

The present nutritional composition preferably also provides omega-9 (n-9) fatty acid (preferably oleic acid, 18:1), to provide sufficient nutrition. Preferably the present composition provides at least 15 wt. % n-9 fatty acid based on the weight of the total fatty acids, more preferably at least 25 wt %. The content of n-9 fatty acids is preferably below 80 wt. %.

The present composition preferably contains vitamin E and/or vitamin C as radical scavengers to prevent oxidation of DPA. Preferably the present composition contains at least 1 mg vitamin E per 100 g dry weight, preferably at least 5 mg. For a nutritional composition the vitamin E content preferably does not exceed 100 mg/100 gram dry weight.

Oligosaccharide

The present composition, besides DPA, preferably comprises indigestible oligosaccharide, preferably at least two different indigestible oligosaccharides. The oligosaccharides preferably influence the mucosal architecture and advantageously influence mucin heterogeneity in the mucus layer, either directly or by changing the intestinal flora. Each different indigestible oligosaccharide is believed to have a different effect on mucus quantity and quality. Moreover, the two distinct oligosaccharides are also able to stimulate quality of mucus as reflected by the degree of sulphation through their synergistic stimulation of SCFA production. It was found by the present inventors that a mixture of two different oligosaccharides synergistically stimulates acetate production. It was also found by the present inventors that mucus production is dependent on acetate production.

In a further aspect, the present composition improves the quality of the intestinal mucus layer. The mucus layer comprises mucins. Mucins are high molecular mass glycoproteins that are synthesized and secreted by goblet cells. They form a gel-like layer on the mucosal surface, thereby improving barrier integrity. The mucus layer comprises different types of mucins, e.g. acid, neutral and sulphonated mucins. An increased heterogeneity of the mucus layer is believed to improve barrier functionality.

The present composition is preferably further improved by providing both long- and short-chain oligosaccharides. The supply of different chain lengths results in stimulation of mucus production in different parts of the ileum and colon. The short chain oligosaccharides (typically with a degree of polymerisation (DP) of 2, 3, 4 or 5) stimulate mucin production in the proximal colon and/or distal ileum, while the oligosaccharides with longer chain lengths (preferably with a degree of polymerisation (DP) of more than 5 up to 60) are believed to stimulate mucin production in the more distal parts of the colon.

Even further improvements can be achieved by providing the at least two different oligosaccharides both as short-chain and long-chain oligosaccharides. These preferred embodiments all contribute to further improved barrier integrity throughout the ileum and/or colon.

The present composition preferably contains an oligosaccharide with a degree of polymerisation (DP) of at least 2 saccharide units, which are not or only partially digested in the intestine by the action of acids or digestive enzymes present in the human upper digestive tract (small intestine and stomach), but which are fermentable by the human intestinal flora. The term saccharide units refers to units having a closed ring structure, preferably hexose, e.g. the pyranose or furanose forms. The degree of polymerisation of the oligosaccharide is typically below 60 saccharide units, preferably below 40, even more preferably below 20.

The present composition preferably comprises at least two different oligosaccharides, wherein the oligosaccharides have a homology in saccharide units below about 90%, preferably below 50%, even more preferably below 25%, even more preferably below 5%. The term “homology” as used in the present invention is the cumulative of the percentage of same saccharide units in the different oligosaccharides. For example, oligosaccharide 1 (OL1) has the structure fruc-fruct-glu-gal, and thus comprises 50% fruc, 25% gal and 25% glu. Oligosaccharide 2 (OL2) has the structure fruc-fruc-glu, and thus comprises 66% fruc, 33% glu. The different oligosaccharides thus have a homology of 75% (50% fruc+25% glu).

In a preferred embodiment, the present composition comprises, besides DPA and optionally EPA, DHA and/or AA, galactooligosaccharides and at least one selected from the group consisting of fructooligosaccharides and inulin.

Each of the present oligosaccharides preferably comprises at least 66%, more preferably at least 90% saccharide units selected from the group consisting of mannose, arabinose, fructose, fucose, rhamnose, galactose, β-D-galactopyranose, ribose, glucose, xylose, uronic acid and derivatives thereof, calculated on the total number of saccharide units contained therein.

According to a preferred embodiment the present composition comprises at least one oligosaccharide selected from the group consisting of fructans, fructooligosaccharides, indigestible dextrins, galactooligosaccharides (including transgalactooligosaccharides), xylooligosaccharides, arabinooligosaccharides, glucooligosaccharides, mannooligosaccharides, fucooligosaccharides, acidic oligosaccharides (see below, e.g. uronic acid oligosaccharides such as pectin hydrolysate) and mixtures thereof. Preferably the present composition comprises at least one, preferably at least two, of the oligosaccharides selected from the group consisting of fructooligosaccharides or inulin, galactooligosaccharides and pectin hydrolysate.

For good mucus quantity and quality, the present composition preferably comprises at least one oligosaccharide, which comprises at least 66% galatose or fructose as a saccharide unit. In a preferred embodiment the composition comprises at least one oligosaccharide which comprises at least 66% galatose as a saccharide unit and at least one oligosaccharide which comprises at least 66% fructose as a saccharide unit. In a particularly preferred embodiment, the present composition comprises galactooligosaccharide and an oligosaccharide selected from the group consisting of fructooligosaccharides and inulin. Fructooligosaccharides stimulate sulfomucin production in the distal colon of human flora-associated rats (Kleessen et al, (2003) Brit J Nutr 89:597-606) and galactooligosaccharides stimulate the acid mucin production (Meslin et al, Brit. J. Nutr (1993), 69: 903-912)).

Preferably the weight ratios:

a. (oligosaccharides with DP 2 to 5):(oligosaccharides with DP 6, 7, 8 and/or 9)>1; and
b. (oligosaccharides with DP 10 to 60):(oligosaccharides with DP 6, 7, 8 and/or 9)>1
are both above 1.

Preferably both weight ratios are above 2, even more preferably above 5.

For even further improvement of mucus layer thickness and quality over the whole area of the colon, preferably each of the at least two different oligosaccharides are provided in different chain lengths, preferably at least 10 wt. % of each oligosaccharide based on the total weight of the respective oligosaccharide has a DP of 2 to 5 (i.e. 2, 3, 4 and/or 5) and at least 5 wt. % has a DP between 10 and 60. Preferably at least 50 wt. %, more preferably at least 75 wt. % of the oligosaccharide based on the total weight of that oligosaccharides has a DP between 2 and 10, because these are believed to work throughout in the ileum and proximal and middle parts of the colon.

When in ready-to-feed liquid form, the present composition preferably comprises 0.1 to 100 grams indigestible oligosaccharide per liter, more preferably between 0.5 and 50 grams per liter even more preferably between 1 and 25 grams per liter. A too high content of oligosaccharides may cause discomfort due to excessive fermentation, while a very low content may result in an insufficient mucus layer.

The weight ratio of the at least two different oligosaccharides is preferably between 1 and 10, more preferably between 1 and 5. These weight ratios stimulate mucin production of different types at different sites in the intestine optimally.

The oligosaccharide is preferably included in the present composition according to the invention in an amount exceeding 0.1 wt. %, preferably exceeding 0.2 wt. %, more preferably exceeding 0.5 wt. % and even more preferably exceeding 1 wt. % based on the total dry weight of the composition. The present composition preferably has an oligosaccharide content below 20-wt. %, more preferably below 10-wt. % even more preferably below 5-wt. %.

Addition of nucleotides and/or nucleosides to the present composition further improves gut mucosal barrier function, particularly as it inhibits and/or or reduces the incidence of bacterial translocation and decreases intestinal injury. Hence, the present composition preferably also comprises between 1 and 500 mg nucleosides and/or nucleotides per 100 gram dry weight, even more preferably between 5 and 100 mg.

### Application

The present composition can be advantageously used in a method for improving barrier integrity in mammals, particularly humans. The present composition can also be advantageously used in a method for the treatment or prevention of diseases associated with reduced barrier integrity, said method comprising administering to a mammal the present composition. The present composition is preferably administered enterally, most preferably orally.

The present composition invention does not encompass natural human breast milk. The present composition is preferably obtainable by mixing ingredients, preferably at least one ingredient from plant, animal or fungal origin with one or more other ingredients. Preferably the present composition comprises at least one of plant oil, animal fat, animal protein, plant protein. The term animal here does not include human. Preferably the present composition is a synthetic composition, i.e. not a body fluid obtained from a live animal or human

For the ill and infants, the present composition is preferably combined with complete nutrition, including protein, carbohydrate and fat. The present composition is advantageously administered to infants with the age between 0 and 2 years. The composition is preferably administered to patients which suffer from an impaired barrier integrity and healthy patients with the risk of developing an impaired barrier function. The present composition is advantageously used in a method for providing the nutritional requirements of a premature infant (an infant born before 37 weeks gestation).

The present composition can also be advantageously used in a method for treatment and/or prevention of intestinal damage by administering the present composition to the patient prior to or after a medical treatment, which may cause intestinal damage. Such medical treatment may for example be surgery or enteral medicine treatment, particularly treatments with antibiotic, analgesic, NSAID and/or chemotherapeutic agents.

The present composition can also be advantageously used to treat or prevent diseases wherein intestinal barrier disruption is underlying the development of the course of the disease, e.g. in a method for the treatment or prevention of chronic inflammatory diseases, particularly inflammatory bowel disease (IBD), irritable bowel syndrome (IBS), celiac disease, pancreatitis, hepatitis, arthritis or diabetes. Furthermore, the invention can be used in a method for providing nutrition to patients which have undergone or are undergoing abdominal surgery and patients that experience postoperative dysfunction of the gut and/or malnourished patients.

In a further embodiment of the invention the present composition is advantageously administered to patients suffering from acquired immune deficiency syndrome (AIDS) and/or patients which are infected with the human immunodeficiency virus (HIV), e.g. in a method for the treatment of AIDS and/or HIV infection. Said method comprises the oral administration of the present composition, preferably combined with nutrients selected from the group consisting of carbohydrate, protein and fat.

Furthermore, the invention can also be used to treat or prevent complications resulting from reduced barrier integrity, particularly in a method for the treatment and/or prevention of diarrhea, particularly infant diarrhea. Due to the reduced incidence in infant diarrhea, the present composition can also be advantageously used to reduce diaper rash.

Administering the present composition reduces passage of dietary and microbial antigens, particularly food allergens, from the intestinal lumen into the mucosal or systemic circulation, and hence can be advantageously used in a method for the treatment or prevention of allergy and/or allergic reaction, particularly in a method for the treatment or prevention of food allergy (e.g. allergic reaction resulting from the ingestion of foodstuff), atopic dermatitis and/or asthma.

Since the barrier function of newborns has not been fully developed, the present composition can be advantageously administered to young infants, i.e. infants with the age between 0 and 6 months. The composition may be administered to the infant in the form of an infant formula or admixed with human milk. Hence the present invention also provides a food formula comprising human milk and the present composition. The compositions including human milk and the present composition are particularly suitable for feeding premature infants. The present composition can be suitably used in a method for the treatment and/or prevention of necrotizing enterocolotis (NEC), particularly in premature infants.

The present composition is preferably provided as a packaged powder or packaged ready-to-feed formula. To prevent spoilage of the product, packaging size of ready-to-feed formula preferably does not exceed one serving, e.g. preferably does not exceed 500 ml; and packaging size of the present composition in powder form preferably does not exceed 250 servings. Suitable packaging sizes for the powder are 2000 grams or less, preferably 1000 grams or less.

The packaged products provided with labels that explicitly or implicitly direct the consumer towards the use of said product in accordance with one or more of the above or below purposes, are encompassed by the present invention. Such labels may for example make reference to the present method for preventing allergic reaction to food allergens by including wording like “reduced food sensitivity”, “improving intestinal tolerability”, “improved food tolerance” or similar wording. Similarly, reference to the present method for treating and/or preventing allergy may be made by incorporating terminology equivalent to “improved resistance” or “reduced sensitivity”. The packaging may also contain indications for improved barrier integrity such as “improved gut health”, “supports development of healthy intestinal function”, “strengthens intestinal defense”, “fortifies healthy gut function”, or similar terminology. This is encompassed by the present patent.

### Food Compositions

It was found that the present composition can be advantageously applied in food, such as baby food and clinical food. Such food preferably comprises lipid, protein and carbohydrate and is preferably administered in liquid form. The term “liquid food” as used in the present invention includes dry food (e.g. powders) which are accompanied with instructions as to admix said dry food mixture with a suitable liquid (e.g. water).

Hence, the present invention also relates to a nutritional composition which preferably comprises between 5 and 50 en % lipid, between 5 and 50 en % protein, between 15 and 90 en % carbohydrate and the present combination of oligosaccharides and LC-PUFA's. Preferably the present nutritional composition preferably contains between 10 and 30 en % lipid, between 7.5 and 40 en % protein and between 25 and 75 en % carbohydrate (en % is short for energy percentage and represents the relative amount each constituent contributes to the total caloric value of the preparation).

Preferably the present composition comprises a combination of vegetable lipid and at least one oil selected from the group consisting of fungal oil, fish oil, seal oil, algal oil or bacterial oil is used.

The proteins used in the nutritional preparation are preferably selected from the group of non-human animal proteins (such as milk proteins, meat proteins and egg proteins), vegetable proteins (such as soy protein, wheat protein, rice protein, and pea protein), free amino acids and mixtures thereof. Cow milk derived nitrogen source, particularly cow milk protein such as casein and whey proteins are particularly preferred.

A source of digestible carbohydrate may be added to the nutritional formula. It preferably provides about 40% to about 80% of the energy of the nutritional composition. Any suitable (source of) carbohydrate may be used, for example sucrose, lactose, glucose, fructose, corn syrup solids, and maltodextrins, and mixtures thereof.

The present composition is preferably used as an infant formula and preferably contains 7.5 to 12.5 energy % protein; 40 to 55 energy % carbohydrates; and 35 to 50 energy % fat. As the present composition is suitably used to reduce the allergic reaction in an infant, the protein of the infant formula is preferably selected from the group consisting of hydrolyzed milk protein (e.g. hydrolyzed casein or hydrolyzed whey protein), vegetable protein and/or amino acids. The use of these proteins further reduced the allergic reactions of the infant.

Stool irregularities (e.g. hard stools, insufficient stool volume, diarrhoea) is a major problem in many babies and ill subjects that receive liquid foods. It was found that stool problems may be reduced by administering the present oligosaccharides in liquid food which have an osmolality between 50 and 500 mOsm/kg, more preferably between 100 and 400 mOsm/kg.

In view of the above, it is also important that the liquid food does not have an excessive caloric density, however still provides sufficient calories to feed the subject. Hence, the liquid food preferably has a caloric density between 0.1 and 2.5 kcal/ml, even more preferably a caloric density of between 0.5 and 1.5 kcal/ml, most preferably between 0.6 and 0.8 kcal/ml.

### EXAMPLES

### Example 1

### Effect of DPA on TER and Flux

Monolayers (MC) of intestinal epithelial cell lines T84 (American Type Culture Collection (ATTC), Manassas, USA) were cultured on transwell filters (Corning, Costar BV, The Netherlands) allowing both mucosal and serosal sampling and stimulation of human intestinal epithelial cells. Two weeks post confluence the monolayers were incubated in the luminal compartment with polyunsaturated fatty acids DPA (7,10,13,16,19-docosapentaenoic acid, 22:5 n3), AA (arachidonic acid; 5,8,11,14-eicosatetraenoic acid), DHA (cis-4,7,10,13,16,19 docosahexaenoic acid), EPA (eicosapentaenoic acid) or control palmitic (C 16:0) acid (Palm) (Sigma, St. Louis, USA). The latter procedure was chosen to mimic the in vivo administration route of the dietary compounds. Cells were incubated with DPA, AA, DHA, EPA, or palmitic acid for 0, 24, 48 and 72 h at 100 μM.

Experiments were performed to evaluate basal barrier integrity and cytokine (IL-4) induced barrier disruption. The epithelial barrier function was determined by measuring the transepithelial resistance (TER, Ω.cm²) by epithelial volt-ohm meter (EVOM; World Precision Instruments, Germany) and permeability for 4 kD FITC dextran (paracellular permeability marker, Sigma, USA). Epithelial permeability for 4 kDa FITC-dextran was determined as follows. Prior to dextran fluxes the medium was refreshed with culture medium without phenol red for one hour followed by addition of 5 μl (stock 100 mg/ml) 4 kDa FITC-dextran to the luminal compartment. After 60 min of incubation 100 μl sample was collected from the serosal compartment and the fluorescent signal measured at excitation wavelength 485 nm and emission 520 nm (FLUOstar Galaxy®, BMG Labtechnologies, USA). FITC-dextran fluxes were calculated as pmol FITC-dextran/cm²/h. Statistical analyses were performed using the ANOVA (SPSS version 10).

Results of the effect of fatty acids (100 μM) on spontaneous barrier integrity and IL-4 mediated barrier disruption after 72 hr incubation are given in Table 1. Table 1 shows that the LC-PUFA DPA, AA, EPA and DHA improve epithelial resistance both under basal as well as under barrier disruptive conditions. In contrast the control experiments show that palmitic acid was not effective. Also increase of permeability caused by incubations with cytokines was effectively reduced by DPA, AA, EPA and DHA and not palmitic acid (PA).

TABLE 1

| Ingredient | Resistance (TER) | Resistance (TER) | Permeability (Flux) |
|---|---|---|---|
| (LC-PUFA) | Basal | Disrupted by IL-4 | Increased by IL-4 |
| Control | 490 ± 55 | 281 ± 7 | 514 ± 109 |
| Palmitic acid | 496 ± 50 | 272 ± 11 | 646 ± 106 |
| DPA | 605 ± 27 | 368 ± 4 | 216 ± 123 |
| DHA | 584 ± 50 | 328 ± 7 | 304 ± 75 |
| AA | 634 ± 29 | 337 ± 13 | 273 ± 76 |
| EPA | 584 ± 7 | 375 ± 9 | 236 ± 145 |

These results are indicative for the advantageous use of DPA, EPA, DHA and AA, and in particularly DPA in the composition according to the present invention and for use in a method according to the present invention, e.g. in a method for improving barrier integrity.

### Example 2

### Effect of Oligosaccharides on Acetate Production

Micro-organisms were obtained from fresh faeces from bottle fed babies. Fresh faecal material from babies ranging 1 to 4 month of age was pooled and put into preservative medium within 2 h. As substrate either prebiotics (TOS; TOS/inulin (HP) mixture in a 9/1 (w/w) ratio; inulin; oligofructose (OS)/inulin mixture in a 1/1 (w/w) ratio, or none (blanc) were used. See also the examples in applicants' co-pending European application no. 04748674.1).

The results show that a mixture of two different oligosaccharides (TOS/Inulin), wherein the two distinct oligosaccharides have a homology in monose units below 90 and a different chain length results in a significantly and synergistically increased amount of SCFA (particularly acetate) per gram fiber than single components. The results also show that the addition of a combination of TOS/Inulin favored a higher proportion of the beneficial acetate. The acetate production in vivo translates to improved mucus production by goblet cells and a measure for intestinal mucus layer thickness (see example 3). These results are indicative for the advantageous use of the present composition.

### Example 3

### Effects of SCFA on Mucus Production

Monolayers of intestinal epithelial T84 were incubated with the short chain fatty acids acetate, proprionate and butyrate (SCFA, Merck, USA) to compare the stimulatory effect of SCFA on MUC-2 expression basal MUC-2 expression levels were deducted. See also the examples in applicants' co-pending European application no. 04748674.1).

The results show that there is a differential effect of SCFA (acetate, proprionate, butyrate) on MUC-2 expression in intestinal epithelial cells (MC T84) and epithelial-mesenchymal cell co-cultures (CC T84). The results also show that acetate is more potent in stimulating MUC-2 expression (mucus production) as compared to propionate and butyrate. Hence, the present combination of oligosaccharides (which was shown to increase acetate production (see example 2)) is particularly useful for stimulating mucus production and can be advantageously used in a method for stimulating barrier integrity.

These results are indicative for the advantageous use of EPA, DHA, DPA and/or AA, in particular DPA, in the composition according to the present invention and for use in a method according to the present invention, i.e. in a method for improving barrier integrity. These results further support the synergistic effects of the present combination of fatty acids and indigestible oligosaccharides.

### Example 4

### Conversion of DPA to EPA in Epithelial Intestinal Cells

Monolayers (MC) of intestinal epithelial cell lines T84 were prepared as described above, and analyzed on fatty acid composition. Fatty acid extraction of the collected T84 cells was performed according to the methods described by Blight, E. G. & Dyer, W. J., 1959 (Can J Med Sci 37: 911-917), using C19:0 as internal standard. In short, under nitrogen atmosphere, lipids were extracted from 1 ml epithelial membrane suspension by adding 2 ml methanol, 0.9 ml EDTA solution (1 g/100 ml MQ) and 1 ml dichloromethane. SPE (solid phase extraction) was used to separate the phospholipids from the other lipids in the extract. After separation, the phospholipids were methylated using 14% BF₃ in methanol for 1 h, according to the methods published by Morrison, W. R. & Smith, L. M., 1964 (J Lipid Res 53: 600-608). After hexane extraction, the fatty acid methyl esters were dissolved in iso-octane and quantified by gas chromatography with capillary column (50 m×0.25 mm, CP-SIL88-fame). The area under the peak was automatically integrated (sensitivity 500 μg/L).

PUFA are incorporated into the intestinal epithelial membrane phospholipids. Table 2 shows that supplementation with fatty acids results in incorporation of all the individual PUFA. After incubation with α-linolenic acid (18:3, n3; ALA), EPA and DHA, the levels of each of these individual fatty acids were incorporated. Surprisingly, when the cells were incubated with DPA, not only DPA was incorporated, but also the EPA level was significantly increased (4.48), showing that supplementation of DPA also enhanced membrane EPA levels (*p<0.015). The DPA stock solution was checked for purity no EPA pollution was found. This observation is indicative for the advantageous use of DPA in the present invention.

TABLE 2

| | | After incubation with: | | | |
|---|---|---|---|---|---|
| | | ALA | EPA | DHA | DPA |
| Fatty acids | C18:3 n-3 (ALA) | 9.64 | 0.00 | 0.07 | 0.04 |
| in | C20:5 n-3 (EPA) | 0.39 | 14.53 | 0.49 | 4.48 |
| membrane: | C22:5 n-3 (DPA) | 0.65 | 1.10 | 0.51 | 6.09 |
| | C22:6 n-3 (DHA) | 1.07 | 0.98 | 6.09 | 0.80 |

### Example 3

### Infant Formula with DPA

A liquid infant nutrition, prepared by admixing 13.9 g powder with water to yield 100 ml final product, said liquid product comprising per 100 ml:

| | |
|---|---|
| Energy: | 66 kcal |
| Protein: | 8 en % |
| Digestible | 44 en % (containing 7.3 g lactose) |
| Carbohydrates: | |
| Lipid: | 48 en % (containing, palm oil, coconut oil, tuna |
| | fish oil, rapeseed oil, sunflower oil and |
| | docopentaenoic acid (Cayman Chemicals, Ann |
| | Arbor, MI, USA) based on total weight of the |
| | lipid 0.2 wt. % DHA; 0.1 wt. % DPA n-3; 0.05 |
| | wt. % EPA; 2.2 wt. % ALA, 0.2 wt. % GLA; |
| | 0.35 wt. % AA, 13 wt. % LA) |
| Fibre: | 0.8 g (containing 0.05 g fructopolysaccharide, |
| | 0.55 g transgalactooligosaccharides. |

Nucleotides:

| | |
|---|---|
| 0.89 mg | Cytidine-5-monophosphate; |
| 0.55 mg | Uridine-5-monophosphate; |
| 0.82 mg | Adenosine-5-monophosphate; |
| 0.20 mg | Guanosine-5-monophosphate; |
| 0.34 mg | Inosine-5-monophosphate. |
| Osmolarity: | 300 mOsmol/l |

The composition further contains choline (6 mg/100 ml) and taurine (6.3 mg/100 ml); minerals and trace elements (including 2 mg zinc/100 ml) and vitamins in amounts in compliance with the international guidelines for infant milk formula.

1-11. (canceled)

12. A composition comprising n-3 docosapentaenoic acid (DPA) and eicosapentaenoic acid (EPA), wherein the ratio of DPA:EPA is equal to or higher than 0.33.

13. The composition according to claim 12 further comprising indigestible oligosaccharides.

14. The composition according to claim 12 comprising 5-60 en % protein, 5-95 en % carbohydrate and 5-75 en % fat.

15. The composition according to claim 13 comprising 5-60 en % protein, 5-95 en % carbohydrate and 5-75 en % fat.

16. The composition according to claim 12, further comprising arachidonic acid and/or docosahexaenoic acid.

17. The composition according to claim 13, further comprising arachidonic acid and/or docosahexaenoic acid.

18. The composition according to claim 14, further comprising arachidonic acid and/or docosahexaenoic acid.

19. The composition according to claim 12, wherein the composition is an infant formula.

20. A method of improving intestinal barrier integrity, stimulating gut maturation, improving barrier function and/or reducing intestinal barrier permeability in a mammal, the method comprising administering to the mammal a composition comprising n-3 docosapentaenoic acid (DPA) and eicosapentaenoic acid (EPA), wherein the ratio of DPA:EPA is equal to or higher than 0.33.

21. The method according to claim 20 in which the mammal is an infant.

22. A method of treating and/or preventing inflammatory bowel disease (IBD), irritable bowel syndrome (IBS), celiac disease, pancreatitis, hepatitis, arthritis, allergy, sepsis, necrotizing enterocolitis, HIV/AIDS, infections, atopic dermatitis or diabetes in a mammal, the method comprising administering to said mammal a composition comprising n-3 docosapentaenoic acid.

23. The method according to claim 22 in which the composition further comprises indigestible oligosaccharides.

24. The method according to claim 22 in which the composition comprises 5-60 en % protein, 5-95 en % carbohydrate and 5-75 en % fat.

25. The method according to claim 23 in which the composition comprises 5-60 en % protein, 5-95 en % carbohydrate and 5-75 en % fat.
